(19) United States
(12) Patent Application Publication
(10) Pub. No.: US 2025/0253054 A1
(43) Pub. Date: Aug. 7, 2025

(54) APPROACH FOR EARLY DETECTION OF DISEASE COMBINING MULTIPLE DATA SOURCES

(71) Applicant: MyOme, Inc., Menlo Park, CA (US)

(72) Inventors: Akash Kumar, Palo Alto, CA (US); Robert MAIER, San Francisco, CA (US); Matthew RABINOWITZ, San Francisco, CA (US); Placede Tshiaba, West Jordan, UT (US); Tate TUNSTALL, San Jose, CA (US)

(21) Appl. No.: 19/188,681

(22) Filed: Apr. 24, 2025

Related U.S. Application Data
(63) Continuation of application No. PCT/US23/77932, filed on Oct. 26, 2023.
(60) Provisional application No. 63/381,198, filed on Oct. 27, 2022.

(51) Int. Cl. G16H 50/30; G16B 40/00; G16H 50/20

(52) U.S. Cl. CPC G16H 50/30; G16B 40/00; G16H 50/20

(57) ABSTRACT

Methods for disease risk assessment using multiple data sources, and computer programs for implementing same.

### CROSS-REFERENCE TO RELATED APPLICATION

This application is a continuation of International Application No. PCT/US2023/077932, filed Oct. 26, 2023, which claims the benefit of U.S. Provisional Application No. 63/381,198, filed on Oct. 27, 2022, which are incorporated herein by reference in their entirety.

### TECHNOLOGICAL FIELD

The present disclosure relates in general to determining disease risk, and more specifically, to methods for determining disease risk to enable early detection of disease.

### BACKGROUND

Early cancer detection (ECD) seeks to identify cancer or precancerous changes in a patient when the disease is most treatable. Approximately 50% of cancers reach an advanced stage before diagnosis, which limits treatment options and decreases survival rates. Early detection of cancer can substantially increase survivability and recent advances in the ability to detect biomarkers associated with cancerous or precancerous tissue hold much promise. However, false positive diagnosis could lead to potentially harmful and unnecessary treatments; therefore, maximizing the accuracy of these methods is essential.

Polygenic risk scores (PRS) have been shown to effectively stratify disease risk in several cancers. Described herein are methods to combine a patient's PRS, along with ECD biomarkers and other relevant information such as family history of relevant cancers to increase the accuracy of early cancer detection and decrease the false positive rate. A similar approach can be used in prediction of other common conditions including coronary artery disease (CAD).

### BRIEF SUMMARY

This invention relates to methods to improve the accuracy of early cancer detection by a priori determination of PRS and subsequent combination with biomarkers to improve accuracy. The approach involves the following steps:
i) Methods to compute an individual's genetic risk inclusive of polygenic risk score (PRS). PRS can be determined using low coverage whole genome sequencing, WGS, or microarray genotypes either at the time of screening or as early as birth;
ii) Methods to measure biomarkers (inclusive of proteins, metabolites); and
iii) (if necessary) updating computed risk to take into account additional clinical variables including age, sex, past history of infectious or environmental exposures (e.g., smoking)

Ultimately this information can determine an individual's risk of currently having cancer (both solid and liquid tumors), heart disease or other common conditions, and allow for more effective interventions. One can improve upon the above approach by also considering genetic correlations between various cancer types; and correlations between different cancer types and the bioanalytical profiles in the blood. Using the disclosed methods, subpopulations who are at higher than average risk for cancer can be identified, which then informs more frequent and/or additional testing, which in turn results in earlier detection and ultimately an increased rates of recovery/survival.

In practice, a patient with borderline or negative early cancer detection (ECD) biomarkers but high polygenic risk for a specific cancer type might be recommended to undergo further testing. In more complex iterations, an individual's risk for multiple cancer types (e.g., Breast, Colon, Pancreatic) which each impact multiple analytes used in early cancer detection (in some cases, like CEA, a biomarker can represent risk for multiple cancers including colon, prostate, lung, thyroid and others) can be leveraged to improve cancer prediction. In a further iteration, an individual's PRS can be used to assign weight to a tumor's origin. This information can then guide additional screening and management of an individual's cancer risk. For example, if an individual has strong breast cancer predisposition genetically and also has ECD signal concerning for cancer, this could lead to a more focused imaging/study like MRI of the breast following a positive ECD result.

The approach may be applied in other contexts as well, such as screening for primary prevention of coronary artery disease. In addition to measuring serum cholesterol and body mass, one can use polygenic risk scores for body mass index (e.g., PRS-BMI) and cholesterol (e.g., PRS-Cholesterol) to inform the predicted risk of coronary artery disease. One can also use genetic risk for an unrelated condition (e.g., BRCA1 pathogenic variant) which is an associated with the CAD-correlated diabetes and associated with an increased risk of complications due to coronary artery. This information can be used to make recommendations for dietary and lifestyle modifications, additional lab work, as well as diagnostic imaging and procedures (e.g., CT-scan or coronary catheterization).

Dietary recommendations could be similar to that of other at-risk patients, but identifying additional genetic risk would lead to a greater probability of following recommendations. Lab work could include more precise (but more expensive) testing usually reserved for more at-risk patients. Continued screening could consist of cancer screening tests that are recommended in the general population, but they could start at a younger age and be conducted at more frequent intervals. A recommended screening frequency can be determined by matching the combined risk of a patient (risk from age plus genetics) to the risk associated with an average person of a specific age. Examples of screening tests that could be recommended on a targeted basis include mammograms for breast cancer and colonoscopies for colon cancer.

Genetic predisposition for cancer is often associated with multiple different types of cancer. This is true for certain monogenic cancer genes, such as BRCA1, as well as for polygenic risk scores, which may capture genome-wide genetic correlations among cancer types. In addition to that, bioanalytical blood profiles and clinical data on cancer risk factors may be correlated with each other and with polygenic risk scores for cancer. Modeling the joint probability distribution of these different modalities makes it possible to improve the accuracy of early cancer detection.

### BRIEF DESCRIPTION OF THE FIGURES

Having described certain example embodiments in general terms above, reference will now be made to the accompanying drawings, which are not necessarily drawn to scale. Some embodiments may include fewer or more components than those shown in the figures.

FIG. 1 illustrates example relationships between a predictive value for a test (PPV) and the prevalence of a cancer in testing populations, in accordance with example embodiments described herein.

FIGS. 2A-2E illustrate example simulations architectures which use PRSs and biomarkers for determining early disease detection for various diseases, in accordance with some example embodiments described herein.

FIGS. 3A-3C illustrates example distributions of PRS and biomarkers as generated by simulations and used for early detection of disease, in accordance with some example embodiments described herein.

FIG. 4 illustrates a schematic block diagram of example circuitry embodying a device that may perform various operations in accordance with example embodiments described herein.

### DETAILED DESCRIPTION

Some example embodiments will now be described more fully hereinafter with reference to the accompanying figures, in which some, but not necessarily all, embodiments are shown. Because inventions described herein may be embodied in many different forms, the invention should not be limited solely to the embodiments set forth herein; rather, these embodiments are provided so that this disclosure will satisfy applicable legal requirements.

Cancer can be treated more effectively when it is detected in earlier stages. Subpopulations that are at higher than average risk can be identified, for whom the cost-benefit tradeoff justifies more frequent testing compared to the general population. This can ultimately increase survival rates and lead to more effective resource allocation.

From an individual's clinical whole genome sequence (WGS), one can generate a bank of polygenic models covering many serious diseases: breast cancer (BC), Jung, prostate and colorectal cancer (CRC), cardio-vascular disease, type II diabetes, stroke, Alzheimer's, liver and kidney disease. These models include the action of tens of thousands of variants in the genome rather than just rare variants of a few genes. The polygenic models can be combined with age, family history and clinical data, and other analytes, as available, to produce Integrated Risk Scores (IRS). These can be clinically reported so that screening and interventions can be planned based on the IRS, reducing healthcare costs, improving outcomes, and optimizing the utility of Multi-Cancer Early Detection (MCED) tests. IRS can be continually optimized across diverse ethnicities, with additional clinical data and analytes such as methylation and new modeling methods such as Deep Neural Networks (DNNs) to capture signaling pathways that cause disease.

A scalable solution can improve access to appropriate screening and interventional care. It can empower individuals of all ethnicities with access to their WGS and its interpretation to proactively manage their own health. It can help physicians respond to a range of phenotypes and individualize intervention, and better characterize disease risk to target treatment, behavior, and diet, with a likelihood of better compliance as individuals understand their personalized risk incorporating their genetics.

IRS can be improved by at least four approaches: 1) enhanced analysis; 2) incorporating rare variants from WGS; 3) incorporating clinical data and additional analytes; and 4) expanded datasets.

In some cases, models can be augmented with “opaque” machine-learning methods such as Neural Networks and with additional analytes to boost AUC and Odds Ratio per Standard Deviation (OR/SD). For example, in BC, the Tyrer-Cuzic (TC) model can be integrated with PRS to boost Area Under the Receiver Operator Curve (AUC) of IRS for remaining lifetime risk over TC. This can involve, e.g., a fixed-stratified method to co-estimate correlated components like age and family history, calibrating risk of subgroups, checking this calibration using the Hosmer-Lemeshow test, and using subpopulation calibration to create a unified Cox Proportional Hazards Model.

In some cases, OR/SD can be boosted over standard PRS. This can involve, e.g., decomposing genomes into ethnic subcomponents, finding the optimal PRSs for each subcomponent, weighting SNPs using HapMap data and SNP ORs for multiple ethnicities as well as functional genomics, and ensemble methods to combine PRSs for the same and associated phenotypes. In some cases, by incorporating Neural Networks (NNs) and Deep NNs (DNNs) to model gene nonlinear coupling and to predict gene expression based on genetic motifs, AUC of linear PRS in BC can also be boosted. These methods can be applied to many diseases.

In some cases, small Copy Number Variants (CNVs) and structural variants (SVs) can be detected alongside single-nucleotide variants (SNVs) from WGS, emulating the performance of multi-protocol genetic panel tests and validating with an orthogonal long-read sequencing method. In some cases, rare disease-associated Loss of Function (LoF) or missense variants can be identified, using ensemble methods and affected genes weighted using public disease-association data. In some cases, reports can be produced that include sets of pharmacogenomic relevant genes to improve compliance with personalized drug and dosing recommendations.

Additional analytes impacting risk can include methylation, mRNA, miRNA, protein, and clinical phenotypes such as blood counts and metabolomics. Methylation from blood, in particular, shows promise as a stable analyte capturing much of the epigenetic effect. Furthermore, methylation can augment AUC of CVD IRS. Similar multiethnic curation are adopted for multiple diseases. In some cases, multiethnic datasets can be curated to enhance polygenic performance. Similar multiethnic curation can be adopted for other diseases. In some cases, a WBS-based “Wellness Test,” can be offered. In some cases, patients can enter personal clinical and family history data to improve their own test performance and actionability.

Data can be pooled to examine the primary outcomes in cases vs controls in each disease for: incidence reduction; earliness of detection; changes in compliance with screening; and interventions. Data can be pooled across enriched and unenriched cohorts. These measures may be combined with kidney and CVD to achieve power if needed. Sensitivity and specificity can also be evaluated for each cancer individually, and for annual MCED tests for high-risk subjects. The health economic model for IRS testing can be generated both with and without MCED. A goal is to show sufficient utility and economic benefit for guideline changes and insurance reimbursement.

### Definition of Certain Terms

Technical and scientific terms used herein have the meanings commonly understood by one of ordinary skill in the art to which the present invention pertains, unless otherwise defined. Materials to which reference is made in the following description and examples are obtainable from commercial sources, unless otherwise noted.

The terms “computer-readable medium” and “memory” refer to non-transitory storage hardware, non-transitory storage device or non-transitory computer system memory that may store computer-executable instructions or software programs that may be accessed by a controller, a microcontroller, a computational system or a module of a computational system. A non-transitory computer-readable medium may be accessed by a computational system or a module of a computational system to retrieve and/or execute the computer-executable instructions or software programs stored on the medium. Exemplary non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM), and the like.

The term “computing device” may refer to any computer embodied in hardware, software, firmware, and/or any combination thereof. Non-limiting examples of computing devices include a personal computer, a server, a laptop, a mobile device, a smartphone, a fixed terminal, a personal digital assistant (“PDA”), a kiosk, a custom-hardware device, a wearable device, a smart home device, an Internet-of-Things (“IoT”) enabled device, and a network-linked computing device.

### Example Implementing Apparatuses

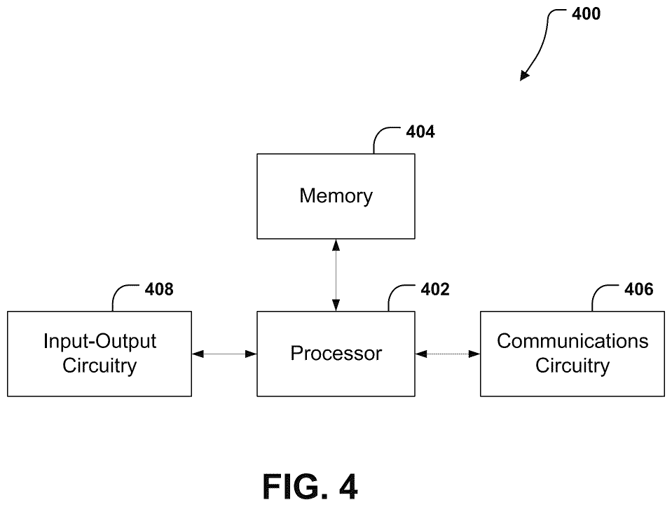

FIG. 4 illustrates an apparatus 400 that may comprise an example system that may implement example embodiments described herein. The apparatus may include processor 402, memory 404, communications circuitry 406, and input-output circuitry 408, each of which will be described in greater detail below, along with and any number of additional hardware components not expressly shown in FIG. 4. While the various components are only illustrated in FIG. 4 as being connected with processor 402, it will be understood that the apparatus 400 may further comprises a bus (not expressly shown in FIG. 4) for passing information amongst any combination of the various components of the apparatus 400. The apparatus 400 may be configured to execute various operations described above, as well as those described below in connection with FIG. 4.

The processor 402 (and/or co-processor or any other processor assisting or otherwise associated with the processor) may be in communication with the memory 404 via a bus for passing information amongst components of the apparatus. The processor 402 may be embodied in a number of different ways and may, for example, include one or more processing devices configured to perform independently. Furthermore, the processor may include one or more processors configured in tandem via a bus to enable independent execution of software instructions, pipelining, and/or multithreading. The use of the term “processor” may be understood to include a single core processor, a multi-core processor, multiple processors of the apparatus 400, remote or “cloud” processors, or any combination thereof.

The processor 402 may be configured to execute software instructions stored in the memory 404 or otherwise accessible to the processor (e.g., software instructions stored on a separate storage device). In some cases, the processor may be configured to execute hard-coded functionality. As such, whether configured by hardware or software methods, or by a combination of hardware with software, the processor 402 represent an entity (e.g., physically embodied in circuitry) capable of performing operations according to various embodiments of the present invention while configured accordingly. Alternatively, as another example, when the processor 402 is embodied as an executor of software instructions, the software instructions may specifically configure the processor 402 to perform the algorithms and/or operations described herein when the software instructions are executed.

Memory 404 is non-transitory and may include, for example, one or more volatile and/or non-volatile memories. In other words, for example, the memory 404 may be an electronic storage device (e.g., a computer readable storage medium). The memory 404 may be configured to store information, data, content, applications, software instructions, or the like, for enabling the apparatus to carry out various functions in accordance with example embodiments contemplated herein.

The communications circuitry 406 may be any means such as a device or circuitry embodied in either hardware or a combination of hardware and software that is configured to receive and/or transmit data from/to a network and/or any other device, circuitry, or module in communication with the apparatus 400. In this regard, the communications circuitry 406 may include, for example, a network interface for enabling communications with a wired or wireless communication network. For example, the communications circuitry 406 may include one or more network interface cards, antennas, buses, switches, routers, modems, and supporting hardware and/or software, or any other device suitable for enabling communications via a network. Furthermore, the communications circuitry 406 may include the processing circuitry for causing transmission of such signals to a network or for handling receipt of signals received from a network.

The apparatus 400 may include input-output circuitry 408 configured to provide output to a user and, in some embodiments, to receive an indication of user input. It will be noted that some embodiments will not include input-output circuitry 408, in which case user input may be received via a separate device. The input-output circuitry 408 may comprise a user interface, such as a display, and may further comprise the components that govern use of the user interface, such as a web browser, mobile application, dedicated client device, or the like. In some embodiments, the input-output circuitry 408 may include a keyboard, a mouse, a touch screen, touch areas, soft keys, a microphone, a speaker, and/or other input/output mechanisms. The input-output circuitry 408 may utilize the processor 402 to control one or more functions of one or more of these user interface elements through software instructions (e.g., application software and/or system software, such as firmware) stored on a memory (e.g., memory 404) accessible to the processor 402.

In some embodiments, various components of the apparatus 400 may be hosted remotely (e.g., by one or more cloud servers) and thus not all components must reside in one physical location. Moreover, some of the functionality described herein may be provided by third-party circuitry. For example, apparatus 400 may access one or more third-party circuitries via any sort of networked connection that facilitates transmission of data and electronic information between the apparatus 400 and the third-party circuitries. In turn, the apparatus 400 may be in remote communication with one or more of the components describe above as comprising the apparatus 400.

As will be appreciated based on this disclosure, some example embodiments may take the form of a computer program product comprising software instructions stored on at least one non-transitory computer-readable storage medium (e.g., memory 404). Any suitable non-transitory computer-readable storage medium may be utilized in such embodiments, some examples of which are non-transitory hard disks, CD-ROMs, flash memory, optical storage devices, and magnetic storage devices. It should be appreciated, with respect to certain devices embodied by apparatus 400 as described in FIG. 4, that loading the software instructions onto a computing device or apparatus produces a special-purpose machine comprising the means for implementing various functions described herein.

Having described specific components of the apparatus 400, example embodiments are described below.

### Example Operations

### Relationship Between IRS Threshold and Sensitivity, PPV, and Specificity

Assume the IRS is normalized to 0 mean and Standard Deviation (SD) 1, and the Odds Ratio (OR) per SD is r. The probability of a positive for disease at IRS value x is p(x)=Cr^{x} where C is some constant. Integrating over all possible values of x and associated probabilities should reproduce the population incidence, p.

$$p = \frac{1}{\sqrt{2 π}} {\int }_{- ∞}^{∞} {Cr}^{x} {e}^{- \frac{1}{2} {x}^{2}} dx = {Ce}^{\frac{1}{2} {( ln ( r ) )}^{2}} , hence C = {pe}^{- \frac{1}{2} {( ln ( r ) )}^{2}}$$

If subjects are flagged that are above some PRS threshold value t, the probability P(t) that a subject is both above some PRS threshold value t and is positive for disease can be found:

$$P ( t ) = \frac{1}{\sqrt{2 π}} {\int }_{t}^{∞} p ( x ) {e}^{- \frac{1}{2} {x}^{2}} dx = \frac{{pe}^{- \frac{1}{2} {( ln ( r ) )}^{2}}}{\sqrt{2 π}} {\int }_{t}^{∞} {r}^{x} {e}^{- \frac{1}{2} {x}^{2}} dx = p ( 1 - normcdf ( t - a ) )$$

Where normcdf is the normal cumulative distribution. Similarly, the probability N(t) that a subject is both above some PRS threshold value t and is negative for disease is:

$$N ( t ) = \frac{1}{\sqrt{2 π}} {\int }_{t}^{∞} ( 1 - p ( x ) {e}^{- \frac{1}{2} {x}^{2}} dx = 1 - normcdf ( t ) - p ( 1 - normcdf ( t - a ) )$$

The sensitivity at threshold t is given by:

$$sensitivity ( t ) = \frac{P ( t )}{p} = 1 - normcdf ( t - ln ( r ) )$$

So, given population incidence p and PRS with OR/SD r, a threshold t can be set to achieve a certain sensitivity:

$$t = ln ( r ) + norminv ( 1 - sensitivity )$$

This gives a Positive Predictive Value (PPV) of:

$$P P V ( t ) = \frac{P ( t )}{P ( t ) + N ( t )} = \frac{p ( 1 - normcdf ( 1 - ln ( r ) ) )}{1 - n o r m c df ( t )}$$

And a specificity of:

$$specificity ( t ) = \frac{1 - p - N ( t )}{1 - p} = \frac{n o r m c d f ( t ) - p ( n o r m c d f ( t - ln ( r ) ) )}{1 - p}$$

Approximating Changes in Sensitivity and Specificity from Changes in AUC

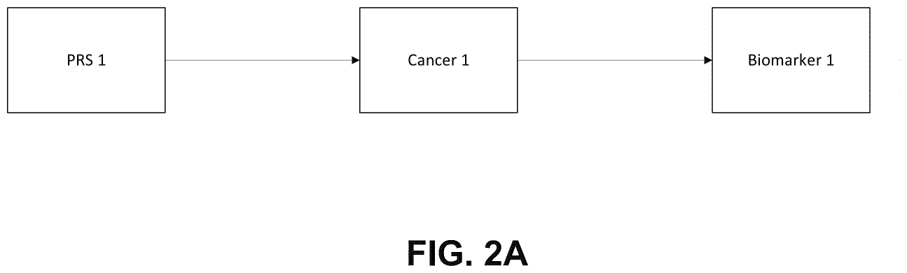

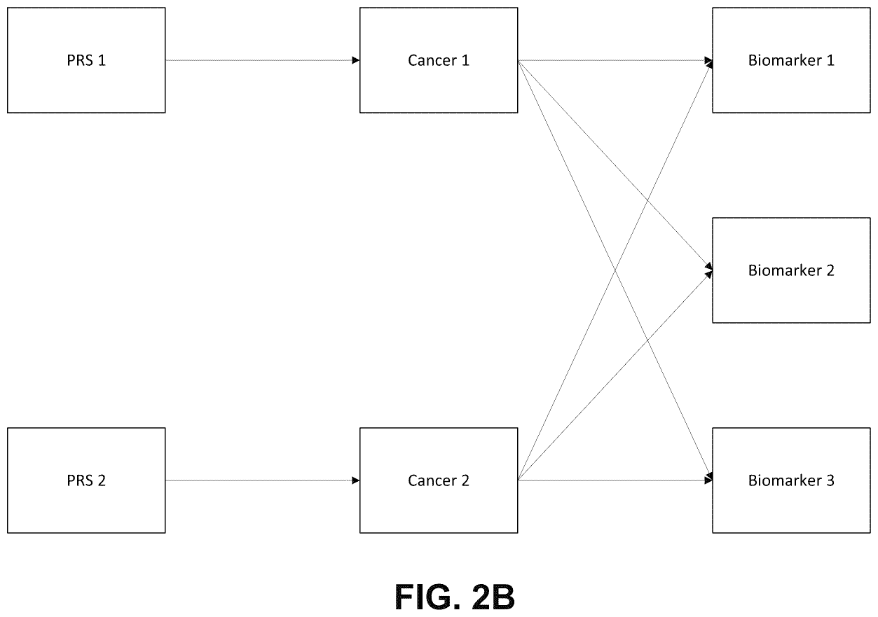

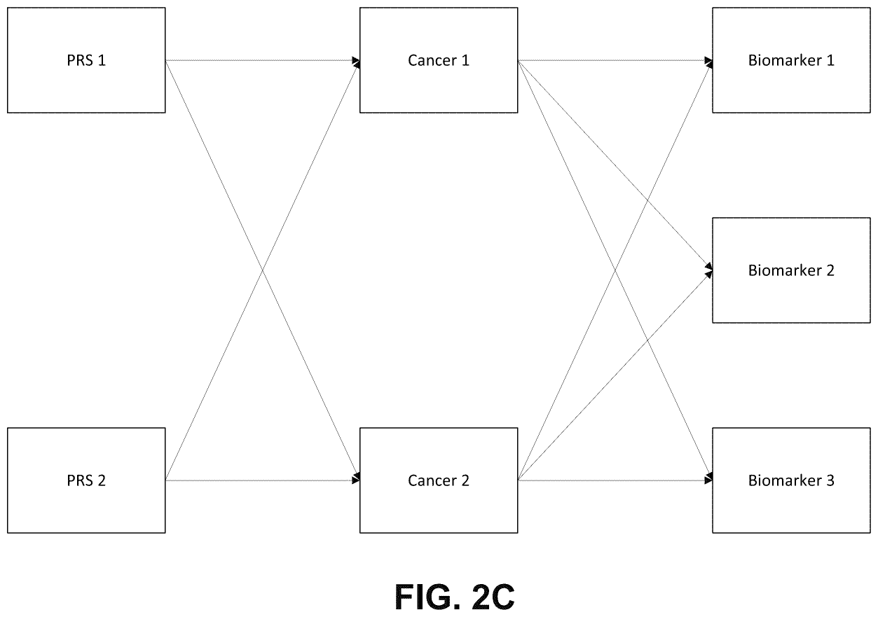

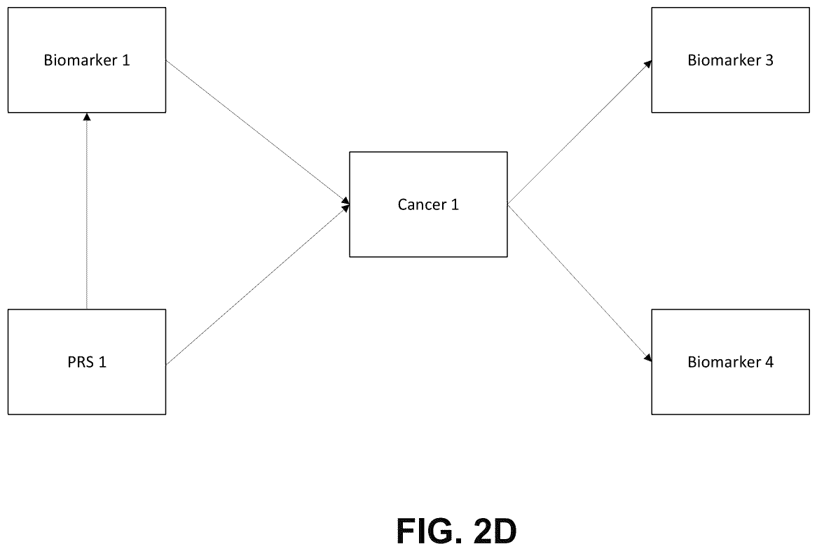

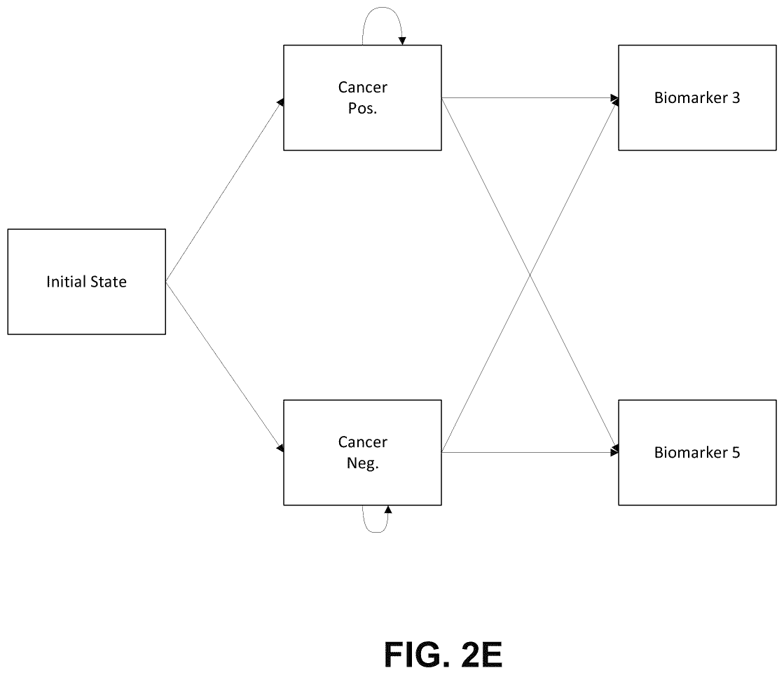

When the shape of the ROC curve is not known, but the change in screening performance based on a change in AUC needs to be approximated, further approximations need to be made. Assume that the ROC curve is symmetric around the x=1−y or specificity=sensitivity line, which represents the average case, and consists of two lines, as shown in FIG. 2. Based on the geometry of these assumptions, it can be shown that A₀=Sens₀=Spec₀ and A₁=Sens₁=Spec₁. When AUC is A₀, assuming an operating point at sensitivity Sens₂, then

$${Spec}_{2} = 1 + \frac{S e n {s}_{2} ( {A}_{0} - 1 ) + {A}_{0}}{{A}_{0}}$$

If the AUC is improved to A₁, and keeping specificity the same while improving sensitivity, the new Sensitivity achievable is

$${Sens}_{2} = \frac{{A}_{1} ( 1 - S p e {c}_{2} )}{1 - {A}_{1}}$$

Working with a PRS

A PRS can be used to select individuals most at risk and recommend them for continued screening. In this scenario, patients are pre-assigned a risk level using PRS, then individuals deemed high risk for the target condition can be recommended for continued monitoring. By testing individuals more likely to develop disease, the precision of the test is increased. The following equation shows this relationship using a cancer screening test as an example:

$$P ( Cancer \vert {Test}_{p o s} ) = \frac{P ( {Test}_{p o s} \vert Cancer ) P ( Cancer )}{P ( {Test}_{p o s} )} \qquad ( 1 )$$

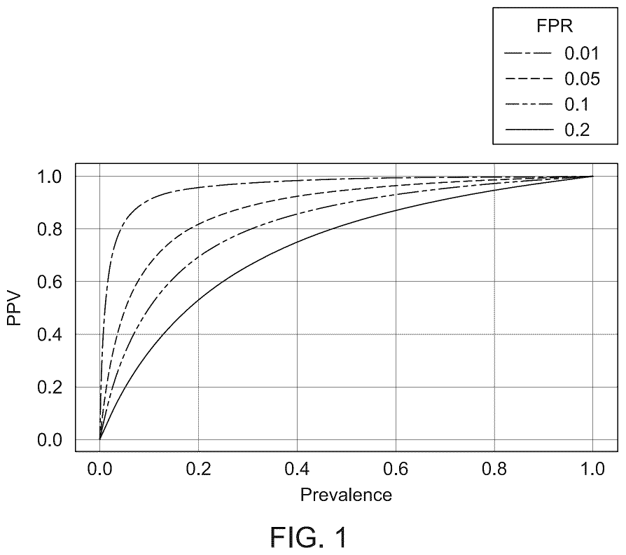

The left-hand side shows the precision, or positive predictive value of the test (PPV). As the prevalence of cancer in the testing population increases as determined by the PRS (P(Cancer), the PPV of the test increases. This phenomenon is further illustrated in FIG. 1, which shows this relationship for a test with a range of false positive rates (FPR). For tests with low FPR, using PRS to focus on patients with the highest risk of disease can result in substantial improvements in PPV.

This approach can prioritize individuals to receive early cancer detection, therefore applying ECD only to the individuals at higher risk, resulting in a more accurate estimate of each individual's risk of cancer, and an increase in the precision of the test

Potential Utility: ECD biomarker tests may be more costly than determining a cancer PRS. To maximize the benefit of the ECD biomarker test, it is useful to identify those patients for whom the ECD biomarker test results are most likely to be clinically actionable. Patients with a high polygenic risk for cancer might benefit more from an ECD biomarker test than patients with a low polygenic risk for cancer.

### Combining PRS and ECD Signals to Reduce False Positives

In addition to stratifying risk, PRS can be combined with an existing ECD test to reduce the false positive rate of a test. The expected false positive rate for detecting cancer was derived using ECD biomarkers only and then using the reduction in the false positive rate that results from combining ECD biomarkers plus PRS. In this example, it was assumed that a population of control samples (D) and a population of patients with cancer (D) possessed normal distributions having equal standard deviations for an ECD biomarker signal X₁. The mean of the population of cancer patients was offset against the mean of the control population, such that the control population has an effective mean of 0 and the mean of the population of cancer patients has an effective mean of m₁. Accordingly, the probability of having cancer given an ECD biomarker signal X₁ can be defined as follows:

$$P ( D \vert {X}_{1} ) = \frac{P ( {X}_{1} \vert D ) P ( D )}{P ( {X}_{1} )} \qquad ( 2 )$$

$$and$$

$$P ( \underline{D} \vert {X}_{1} ) = \frac{P ( {X}_{1} \vert \underline{D} ) P ( \underline{D} )}{P ( {X}_{1} )} \qquad ( 3 )$$

It was assumed that the overall probability of a patient having cancer is equal to the overall probability of patient being cancer free (i.e., P(D)=P(D)). The threshold, t₁, above which an ECD biomarker signal X₁ is considered to be indicative of cancer was set at the X₁ level of m₁/2, where the probability of cancer is equal to the probability of non-cancer for the same X₁ signal (i.e., P(D)=P(D)). Accordingly, the above equations can be solved to show that at t₁:

$$P ( {X}_{1} \vert \underline{D} ) = P ( {X}_{1} \vert D ) \qquad ( 4 )$$

The probability of the signal X₁ corresponding to a false positive (i.e., falsely characterizing a control sample as a cancer sample) was then computed from the cumulative distribution function using X₁ as follows:

$$\begin{matrix}Probability of false positive = {\int }_{P ( {X}_{1} \vert D ) < P ( {X}_{1} \vert \underline{D} )}^{i n f} P ( {X}_{1} \vert D ) d {x}_{1} \\ = {\int }_{t}^{inf} P ( {X}_{1} \vert D ) d {x}_{1} \\ = {\int }_{\frac{m 1}{2}}^{inf} \frac{1}{s q r t ( 2 * p i )} {e}^{\frac{- {X}_{1}^{2}}{2}} d {x}_{1}\end{matrix} \qquad ( 5 )$$

### False Positives in Detecting Cancer Using ECD Biomarker Plus PRS

A method of making a cancer/control call from using two signals together (X₁, an ECD biomarker signal and an orthogonal signal, X₂, which may be a PRS) was computationally simulated according to the call scheme shown in Table 1, below:

TABLE 1

| X₁ | X₂ | Combined call |
|---|---|---|
| <m₁/2 | <m₂/2 | Negative |
| >m₁/2 | >m₂/2 | Positive |
| <m₁/2 | >m₂/2 | No call |
| >m₁/2 | <m₂/2 | No call |

The same assumptions made for the distribution of signal X₁, as described above, were made for the distribution for signal X₂. The probability of calling a false positive and the probability of failing to make any call based on the using both distributions according to Table 1 were determined as follows in Table 2, wherein “normcdf” is the normal cumulative distribution function (e.g., as in MATLAB®);

TABLE 2

| Probability | Variable | Calculation |
|---|---|---|
| Probability of False Positive | P_{FPX1} | 1-normcdf(m₁/2) |
| based on X₁ | | |
| Probability of False Positive | P_{FPX2} | 1-normcdf(m₂/2) |
| based on X₂ | | |
| Probability of False Positive | P_{FPX1X2} | P_{FPX1} * P_{FPX2} |
| based on X₁ and X₂ | | |
| Probability of No Call based | P_{NCX1X2} | P_{FPX1}*(1 − P_{FPX2}) + |
| on X₁ and X₂ | | P_{FPX2}*(1 − P_{FPX1}) |

Assuming m₁=6 and m₂=6/sqrt(3), probability values were calculated as follows: P_{FPX1}=0.0013; P_{FPX2}=0.0416; and P_{FPXIX2}=0.000056.

A population of control (D) measurements and a population of cancer measurements were assumed to have the same distributions as in the previous example. A method of making a cancer/control call from mathematically combining the two signals, X₁ and X₂, into a single product (X₁*X₂ or “X₁X₂”) was calculated as follows:

$$P ( D \vert {X}_{1} {X}_{2} ) = \frac{P ( {X}_{1} {X}_{2} \vert D ) P ( D )}{P ( {X}_{1} {X}_{2} )} \qquad ( 6 )$$

$$and$$

$$P ( \underline{D} \vert {X}_{1} {X}_{2} ) = \frac{P ( {X}_{1} {X}_{2} \vert \underline{D} ) P ( \underline{D} )}{P ( {X}_{1} {X}_{2} )} \qquad ( 7 )$$

Assuming again that the overall probability of non-cancer is equal to the overall probability of cancer (i.e.,), then at threshold, t:

$$P ( D \vert {X}_{1} {X}_{2} ) = P ( \underline{D} \vert {X}_{1} {X}_{2} ) \qquad ( 8 )$$

$$P ( D \vert {X}_{1} {X}_{2} ) = P ( {X}_{1} \vert D ) P ( {X}_{2} \vert D ) \qquad ( 9 )$$

$$P ( {X}_{1} {X}_{2} ) = P ( {X}_{1} \vert \underline{D} ) P ( {X}_{2} \vert \underline{D} ) \qquad ( 10 )$$

The joint probability function was then integrated to evaluate the false positive rate, as follows:

$$\begin{matrix}P ( False Positive ) = {\int }_{P ( {X}_{1} \vert D ) P ( {X}_{2} \vert D ) < P ( {X}_{1} \vert \underline{D} ) P ( {X}_{2} \vert \underline{D} )}^{inf} \\ P ( {X}_{1} \vert D ) P ( {X}_{2} \vert D ) {dx}_{1} d {x}_{2} \\ = {\int }_{P ( {X}_{1} \vert D ) P ( {X}_{2} \vert D ) < P ( {X}_{1} \vert \underline{D} ) P ( {X}_{2} \vert \underline{D} )}^{inf} \\ \frac{1}{2 * pi} {e}^{\frac{- ( {X}_{1}^{2} + {X}_{2}^{2} )}{2}} d {x}_{1} d {x}_{2}\end{matrix} \qquad ( 11 )$$

where P(X₁|D)P(X₂|D)<P(X₁|D)P(X₂|D) can be clarified further to:

$$\frac{P ( {X}_{1} \vert D ) P ( {X}_{2} \vert D )}{P ( {X}_{1} \vert \underline{D} ) P ( {X}_{2} \vert \underline{D} )} < 1 \qquad ( 12 )$$

$$1 {e}^{\frac{- ( {X}_{1}^{2} )}{2 {σ}^{2}} + \frac{{( {X}_{1} - {m}_{1} )}^{2}}{2 {σ}^{2}}} * {e}^{\frac{- ( {X}_{2}^{2} )}{2 {σ}^{2}} + \frac{{( {X}_{2} - {m}_{2} )}^{2}}{2 {σ}^{2}}} < 1$$

$$2 {X}_{1} {m}_{1} + 2 {X}_{2} {m}_{2} > {m}_{1}^{2} + {m}_{2}^{2}$$

X₂ was then solved as follows:

$${X}_{2} > \frac{{m}_{1}^{2} + {m}_{2}^{2} - 2 {m}_{1} {X}_{1}}{2 {m}_{2}} \qquad ( 13 )$$

Accordingly, the false positive rate was determined to be:

$$P ( False Positive ) = {\int }_{- inf}^{inf} \frac{{\int }_{{m}_{1}^{2} + {m}_{2}^{2} - 2 {m}_{1} {X}_{1}}^{inf}}{2 {m}_{2}} \frac{1}{2 * pi} {e}^{\frac{- ( {X}_{1}^{2} + {X}_{2}^{2} )}{2}} d {x}_{1} d {x}_{2} \qquad ( 14 )$$

The false positive rate was then able to be empirically calculated using the following MATLAB® code, wherein “sum” is the false positive rate, for different signal means, m₁ and m₂:

```
% variables
n = 2000;
m1 = 6;
m2 = 6/sqrt(3);
lim = 20;
delta = 2*lim/(n−1);
x1_vec = [−lim:delta:lim];
x2_vec = [−lim:delta:lim];
sum = 0;
for x1 = x1_vec
  ind = find( x2_vec > (m1\hat{ }2 + m2\hat{ }2 −2*m1*x1)/(2*m2) );
  for x2 = x2_vec(ind)
    sum = sum + exp(−0.5*(x1\hat{ }2+x2\hat{ }2))*delta\hat{ }2/(2*pi);
  end
end
sum
```

Here, “sum” corresponds to the probability of observing a false positive in this joint probability scenario combining signal mean m1 and the somewhat weaker signal mean m2. The probability of a false positive was determined to be: P(False Positive)=sum=0.00026, whereas the individual probabilities (evaluated in the previous example) were determined to be higher: P_{FPX1}=0.0013 and P_{FPX2}=0.0416.

The simulation demonstrates that combining two independent signals, with one signal having a 3-fold higher variance than the other, can reduce the false positive rate by at least a factor of 5, relative to using either of the signals alone.

Simulation results showing increased specificity for correlated traits are shown in Table 3 below.

TABLE 3

| | y~x1 | | y~x2 | | y~x1 + x2 | |
|---|---|---|---|---|---|---|
| Sample size | Sens | Spec | Sens | Spec | Sens | Spec |
| 1,000 | 0.408 | 0.9413 | 0.18 | 0.96 | 0.744 | 0.9427 |
| 2,000 | 0.122 | 0.9693 | 0.01 | 0.996 | 0.248 | 0.946 |
| 5,000 | 0.0648 | 0.9816 | 0.3984 | 0.9293 | 0.6168 | 0.9314 |
| 20,000 | 0.0994 | 0.9721 | 0.091 | 0.9766 | 0.213 | 0.9502 |
| 50,000 | 0.0159 | 0.9951 | 0.4882 | 0.9258 | 0.6294 | 0.9297 |
| 100,000 | 0.6862 | 0.9370 | 0.0626 | 0.9832 | 0.7104 | 0.9383 |

Simulation of Including PRS with a Single Cancer Biomarker

FIG. 2A depicts an example simulation for a single cancer biomarker. The utility of PRSs is further demonstrated by simulating data and comparing early detection models with and without a PRS component. The following plate diagram illustrates the scenario of the simulation:

Here a PRS is predictive of cancer risk, and cancer status is associated with a biomarker that is used as an indicator of cancer in an early detection test.

The outline of the simulation was as follows:
1. Assume a PRS standardized to N˜(0,1)
2. Simulate cancer status using PRS log odds ratios of 1.5 to 3, consistent with effects found in other diseases (4.5, T1 diabetes; 1.9, prostate cancer; 1.74, breast cancer), and an intercept based on average population risk.
3. Simulate a ECD biomarker as Poisson (λ+k(t))
   a. λ is the baseline value for the biomarker in the uninfected population
   b. k(t) simulates the growth of the biomarker as cancer progresses at time t, simulated using a logistic function
4. The time t since the onset of cancer is modeled as a negative binomial distribution with mean 10 days
5. Simulate cancer status for 500 k individuals
6. Compare a biomarker only model and a model incorporating both the biomarker and PRS

The addition of PRS is most effective when the normal ECD test is underpowered, resulting in up to a 15-point improvement in recall when combined with PRS, and even underpowered PRSs result in an increase in recall as shown in Table 4.

TABLE 4

| | | Recall | | | Precision | | |
|---|---|---|---|---|---|---|---|
| Beta | Biomarker | with | Biomarker | | with | Biomarker | |
| PRS | Growth | PRS | only | delta | PRS | only | delta |
| 1.5 | 0.05 | 0.21 | 0.17 | 0.03 | 0.71 | 0.74 | −0.03 |
| | 0.1 | 0.74 | 0.75 | 0.00 | 0.94 | 0.92 | 0.01 |
| | 0.15 | 0.92 | 0.91 | 0.00 | 0.99 | 0.99 | 0.00 |
| | 0.2 | 0.97 | 0.97 | 0.00 | 0.99 | 0.99 | 0.00 |
| 2 | 0.05 | 0.26 | 0.22 | 0.04 | 0.71 | 0.67 | 0.04 |
| | 0.1 | 0.76 | 0.75 | 0.01 | 0.93 | 0.93 | 0.00 |
| | 0.15 | 0.92 | 0.91 | 0.01 | 0.98 | 0.99 | 0.00 |
| | 0.2 | 0.97 | 0.97 | 0.00 | 1.00 | 0.99 | 0.00 |
| 2.5 | 0.05 | 0.32 | 0.22 | 0.10 | 0.72 | 0.69 | 0.02 |
| | 0.1 | 0.78 | 0.75 | 0.03 | 0.93 | 0.94 | −0.01 |
| | 0.15 | 0.93 | 0.92 | 0.01 | 0.98 | 0.99 | 0.00 |
| | 0.2 | 0.97 | 0.97 | 0.00 | 1.00 | 0.99 | 0.00 |
| 3 | 0.05 | 0.38 | 0.23 | 0.15 | 0.72 | 0.72 | 0.00 |
| | 0.1 | 0.79 | 0.75 | 0.04 | 0.93 | 0.94 | −0.01 |
| | 0.15 | 0.93 | 0.93 | 0.00 | 0.98 | 0.98 | 0.01 |
| | 0.2 | 0.97 | 0.97 | 0.00 | 0.99 | 1.00 | 0.00 |

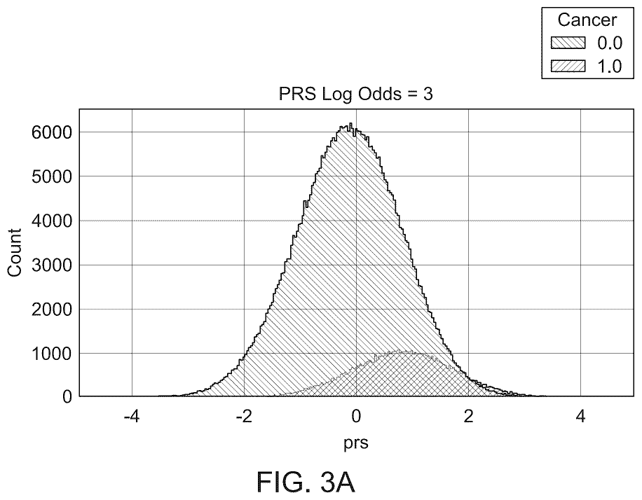

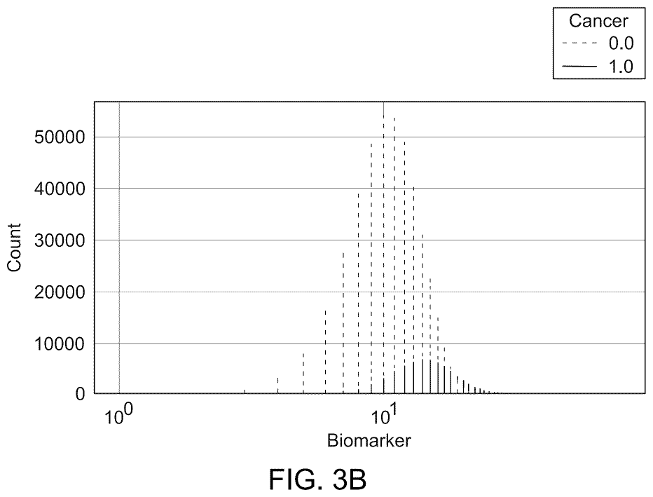

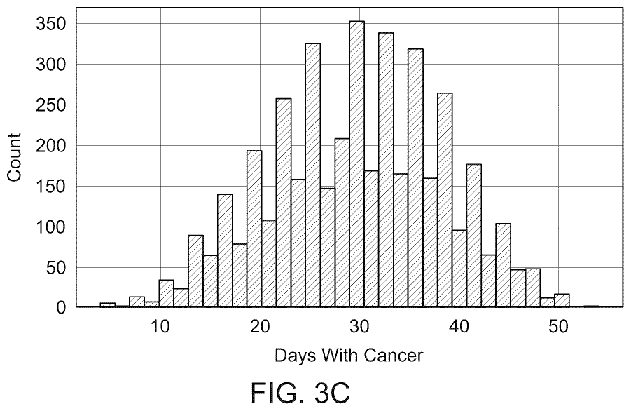

As shown in FIGS. 3A-3B, with a PRS logs odds of 3 and a moderately powered ECD biomarker (see FIG. 3A), there is a 15% increase in recall in the simulations. This PRS is well within the range found empirically, and can be made more powerful by including additional covariates. The simulated biomarker test was moderately powered, where early disease states are difficult to discriminate. This is realistic given most early cancer tests are still under development.

Including PRS can allow for cases to be identified earlier than a biomarker only test. The median case identified by the full model was 6 days earlier than the biomarker only model, and cases missed by the biomarker only model and identified by the full model were earlier stage cancers as shown in FIG. 3C.

In this relatively simple scenario, incorporating PRS into a ECD test results in substantial improvement. More complex scenarios, such as additional biomarkers and correlated PRS, could result in an additional improvement.

### Additional Example 1: Screening for Multiple Cancers

As depicted in FIG. 2B, the simple single cancer/biomarker scenario is extended to include multiple cancers or diseases where PRS for different cancers might be uncorrelated, but biomarkers are correlated. This allows us to use the correlation between biomarkers to increase the power to detect both cancer #1 and #2. Examples of cancer biomarkers which are shared among different cancer types include HER2/neu, Alpha-fetoprotein, and Carcinoembryonic antigen. Predicting PRS for cancers which share biomarkers not only increases the power to detect each cancer type, it may also help differentiate between cancer types.

### Additional Example 2

As depicted in FIG. 2C, the model is further extended to multiple PRS correlated with multiple cancers. Each cancer has a specific biomarker profile (occasionally sharing biomarkers). The correlation of PRSs gives further power to detect cancers. The genetic correlation between cancer types illustrated in this example can increase power to detect both cancer types in addition to the increases in power that come from the shared biomarkers.

### Additional Example 3

As depicted in FIG. 2D, the model is extended to a case where a PRS predicts a biomarker that contributes to disease risk itself, rather than simply being a response. Examples 2 and 3 illustrate cases where tumors trigger the secretion of biomarkers which are subsequently used for diagnosis. However, some biomarkers are causally upstream of tumor development, which affects the joint distributions of biomarkers and polygenic scores, and the statistical modeling techniques required for cancer detection. As such, consideration of these upstream biomarkers may be predicted by the PRS model without direct measurement, thereby yielding a more robust and accurate risk of disease for an individual.

### Additional Modeling:

FIG. 2E depicts a more complex simulation. To model the complex interactions between biomarkers, PRS, and cancer risk, statistical modeling is used to model such interactions. In some embodiments, the statistical modeling uses a Hidden Markov Model (HMM). In some embodiments, the statistical modeling uses a machine learning model, such as a neural network. In FIG. 2E, the black arrows represent a transition between states, and blue arrows represent observations. Biomarker data is observed over time, and PRS contributes to the probability of state transitions.

The Viterbi (dynamic programming) algorithm is then used to compute the most likely state sequence to have generated the observations:

```
generated quantities {
  array[T_unsup] int<lower=1, upper=K> y_star;
  real log_p_y_star;
  {
  array[T_unsup, K] int back_ptr;
  array[T_unsup, K] real best_logp;
  real best_total_logp;
  for (k in 1:K) {
    best_logp[1, k] = log(phi[k, u[1]]);
  }
  for (t in 2:T_unsup) {
    for (k in 1:K) {
    best_logp[t, k] = negative_infinity( );
    for (j in 1:K) {
      real logp;
      logp = best_logp[t − 1, j]
        + log(theta[j, k]) + log(phi[k, u[t]]);
      if (logp > best_logp[t, k]) {
      back_ptr[t, k] = j;
      best_logp[t, k] = logp;
      }
    }
    }
  }
  log_p_y_star = max(best_logp[T_unsup]);
  for (k in 1:K) {
    if (best_logp[T_unsup, k] == log_p_y_star) {
    y_star[T_unsup] = k;
    }
  }
  for (t in 1:(T_unsup − 1)) {
    y_star[T_unsup − t] = back_ptr[T_unsup − t + 1,
```

y_star[T_unsup − t + 1]];

```
  }
  }
}
```

### CONCLUSION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

What is claimed is:

1. A method for determining whether a subject is at increased risk for a disease, the method comprising:
determining a whole genome sequence of the subject;
generating a polygenic risk model based on the clinical whole genome sequence;
applying the polygenic risk model to a subject genotype to generate a polygenic risk score (PRS) for the subject, wherein the polygenic model is associated with a particular disease or disease group;
determining one or more biomarker values for the subject, wherein one or more of the one or more biomarkers are associated with the particular disease or disease group;
determining one or more recommended actions for the subject based at least in part on the PRS score and the one or more biomarker values; and
providing an indication of one or more of the one or more recommended actions for the subject, one or more biomarker values for the subject, or the PRS for the subject.

2. The method of claim 1, further comprising:
assigning the subject to a risk category based on the PRS; and
in an instance the assigned risk category corresponds to a risk category associated with an elevated risk of disease, determining a recommended biomarker action, wherein the recommended biomarker action describes one or more recommended biomarker tests to be performed on the subject and the recommended biomarker action is included in the one or more recommended actions.

3. The method of claim 2, wherein the one or more biomarker values are determined based on results from the one or more recommended biomarker tests.

4. The method of claim 1, wherein a disease status used in the polygenic risk model is determined using a PRS log odds ratio.

5. The method of claim 4, wherein a value of the PRS log odds ratio is based on an average population risk.

6. The method of claim 1, further comprising:
applying an additional polygenic risk model to the subject genotype to generate one or more additional PRSs for the subject, wherein a subset of the one or more biomarker values are associated with an additional particular disease or disease group associated with the additional polygenic risk model.

7. The method of claim 6, wherein one or more of the one or more biomarker values are associated with both the particular disease or disease group associated with the polygenic risk model and the additional particular disease or disease group associated with the additional polygenic risk model.

8. The method of claim 6, wherein the additional particular disease or disease group is also associated with the polygenic risk model.

9. The method of claim 1, wherein applying the polygenic risk model to the subject genotype further comprises:
generating one or more upstream biomarker values for the particular disease or the disease group associated with the polygenic risk model; and
determining the one or more recommended actions for the subject based at least in part on the PRS score, the one or more biomarker values, and the one or more upstream biomarker values.

10. The method of claim 1, further comprising estimating a joint probability distribution of the PRS and the one or more biomarker values.

11. The method of claim 1, further comprising estimating a subset of the one or more biomarker values based on the PRS.

12. The method of claim 1, further comprising:
determining a disease likelihood state for the subject using statistical modeling, wherein the disease likelihood state is indicative of whether the subject is estimated to be positive or negative for the disease or disease group associated with the PRS.

13. The method of claim 12, wherein a hidden Markov model is used for the statistical modeling.

14. An apparatus for determining whether a subject is at increased risk for a disease, the apparatus comprising a processor and a memory storing software instructions that, when executed by the processor, cause the apparatus to perform a method comprising:
determining a whole genome sequence of the subject;
generating a polygenic risk model based on the clinical whole genome sequence;
applying the polygenic risk model to a subject genotype to generate a polygenic risk score (PRS) for the subject, wherein the polygenic model is associated with a particular disease or disease group;
determining one or more biomarker values for the subject, wherein one or more of the one or more biomarkers are associated with the particular disease or disease group;
determining one or more recommended actions for the subject based at least in part on the PRS score and the one or more biomarker values; and
providing an indication of one or more of the one or more recommended actions for the subject, one or more biomarker values for the subject, or the PRS for the subject.

15. A computer program product for determining whether a subject is at increased risk for a disease, the computer program product comprising at least one non-transitory computer-readable storage medium storing software instructions that, when executed, cause an apparatus to perform a method comprising:
determining a whole genome sequence of the subject;
generating a polygenic risk model based on the clinical whole genome sequence;
applying the polygenic risk model to a subject genotype to generate a polygenic risk score (PRS) for the subject, wherein the polygenic model is associated with a particular disease or disease group;
determining one or more biomarker values for the subject, wherein one or more of the one or more biomarkers are associated with the particular disease or disease group;
determining one or more recommended actions for the subject based at least in part on the PRS score and the one or more biomarker values; and
providing an indication of one or more of the one or more recommended actions for the subject, one or more biomarker values for the subject, or the PRS for the subject.
